(19) United States
(12) Patent Application Publication
(10) Pub. No.: US 2022/0096634 A1
(43) Pub. Date: Mar. 31, 2022

(54) METHOD OF MANIPULATING PHAGES WITHIN THE BODY TO ATTACK INFECTIOUS TARGET CELLS

(71) Applicant: David A. Jernigan, Sarasota, FL (US)

(72) Inventor: David A. Jernigan, Sarasota, FL (US)

(21) Appl. No.: 17/490,486

(22) Filed: Sep. 30, 2021

Related U.S. Application Data
(60) Provisional application No. 63/085,507, filed on Sep. 30, 2020.

(51) Int. Cl. A61K 41/00; A61N 5/06; A61K 9/08

(52) U.S. Cl. CPC A61K 41/00; A61K 9/08; A61N 5/062

(57) ABSTRACT

A method and system is disclosed for regulating, manipulating or inducing a change of activity, from lysogenic to lytic activities in native or naturally occurring phages within a subject to treat a bacterial, viral, fungal, or parasitic infection, or other pathological or biologically-physiologically advantageous target in the subject, the method including the steps of identifying the target within the subject and exposing the subject to a treatment electromagnetic signature that is suitable to induce the native phages in the subject to attack the target, or advantageously modify the target pathology or substance. The method can include implanting the treatment electromagnetic signature within an ingestible carrier medium and providing the ingestible carrier medium to the subject.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

This application is a non-provisional of U.S. patent application Ser. No. 63/085,507 filed Sep. 30, 2020 entitled METHOD OF MANIPULATING PHAGES WITHIN THE BODY TO ATTACH INFECTIOUS TARGET CELLS, which is hereby incorporated by reference in its entirety.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND OF THE INVENTION

The present disclosure relates generally to treatment technologies for the treatment of harmful organisms.

More particularly, the present disclosure relates to treatment methods for various diseases, including but not limited to, treatment-resistant tick-borne illness, such as Lyme disease and its co-infections, Staph infections, or any other type of illness. One conventional method of treatment for such diseases is antibiotics. While conventional antibiotic medications have undoubtedly saved countless lives to date, present antimicrobial medications and antibiotics lack precision, often requiring the use of multiple types of antibiotics with known neurotoxic effects that can kill beneficial microbes, damage organs and tissues, and alter normal biochemistry within the patient.

The treatment goal when dealing with infection is to eliminate all of the offending microbial population, while resisting mutation to escape being killed, and with minimal ancillary harm. In human therapeutics, the substantially complete elimination of the pathogen will enable the body to return to a healthy state. In contrast to the effectiveness of phage therapy, antibiotics will most often only kill a percentage of the targeted bacteria, leaving a small percentage now mutated and antibiotic-resistant. Antibiotic-induced dormancy of bacteria, known as bacterial persistor cells, is being recognized as a cause of recurrent and chronic illness from infection. The best that can be hoped for with most conventional antibiotic medication is remission, with the hope that the immune system can keep the remaining bacteria and persistor cells from reemerging when the human organism is under physical, emotional, or environmental stress. The lack of effectiveness of antibiotics, the antibiotic-induced bacterial mutations, as well as people's increasing inability to tolerate natural or prescription antibiotics, is leaving doctors and their patients with few options, leading to extreme and prolonged suffering and premature death. To make matters worse, many natural and prescription treatments put too much of a strain on the already profoundly weakened person.

The introduction of externally-sourced phages into the body, which naturally target and kill specific bacteria, is well-recognized by the scientific community as an effective treatment method to fight bacterial infections. Phages are ubiquitous in nature and are the dominant species on the planet. Phages are viruses that most often selectively infect one specific type of microbe to use as a host, in a parasitic-relationship. For example, Borrelia-phages only infect Borrelia bacteria. Staphylococcus-phages only infect Staphylococcus bacteria, etc. However, introduction of externally-sourced or foreign phages into the body is currently not approved by the many regulatory bodies worldwide, and thus is currently unavailable as a treatment method in many countries.

What is needed then are improvements in methods of treating harmful organisms.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is a novel method of treating bacterial and other types of infections, that enables the activation (induction) of viruses called, phages. These phages are named by the type of host they select. Phages that use mold as a host are called Mycophages. Phages that use bacteria as their host are called bacteriophage. This invention is a novel technology and treatment that induces native-phages to target and kill their host organism, using the technology to determine and apply precise and complex electromagnetic fields (EMF) imprinted or generated within a carrier medium, such as sterile water, hydro-alcoholic solution, saline solution, sugar-base, electromagnetic device, powder, tablet/pill/capsule, or hydro-nanoparticle solution. Such carrier mediums can be delivered on or into the human body or organism using numerous suitable delivery methods as a treatment of the specific infectious microbe within the host subject. While certain carrier mediums and delivery methods have been identified in this disclosure, other equivalent carrier mediums and delivery methods will be readily apparent to one of skill in the art.

Another aspect of the present disclosure is a method of regulating, manipulating or inducing a change of activity, from lysogenic to lytic activities in native or naturally occurring phages within a subject to treat a bacterial, viral, fungal, or parasitic infection, or other pathological or biologically or physiologically advantageous target substance in the subject. The method comprising the steps of: identifying the target within the subject; and exposing the subject to a treatment electromagnetic signature that is suitable to induce the native phages in the subject to attack the target, or advantageously modify the target pathology or substance.

The energy of induction or triggering electromagnetic field signature reproduced in the carrier medium or solution can be capable of stimulating a predetermined phage-type, which already exists within the subject, such as a human, animal, bugs, food substance, water-treatment or other organism which may be exposed or carry infectious target cells, to switch from lysogenic activities to, but not limited to, lytic activities, resulting in the attacking and substantial elimination of their host target cell, or modifying of the target substance within the organism being addressed with phages. The method of the present disclosure only seeks to induce native phages to preferentially switch from lysogenic parasitizing of their host target, to lytic or other beneficial activities, which has been previously determined as a worthy therapeutic goal. The method disclosed herein beneficially does not involve the introduction or use of externally-sourced, or genetically-modified phages.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a front view of one step of an exemplary treatment of the present disclosure including identifying a microbe within a subject and determining a triggering electromagnetic field signature for causing a phage associated with the microbe to attack the microbe via lytic swarming.

FIG. 2 is a perspective diagram of a nanoparticle solution being charged with electromagnetic energy such that the nanoparticle resonates a treatment electromagnetic field having a similar electromagnetic field signature as the triggering electromagnetic field signature

FIG. 3 is a top view of the charged nanoparticle solution of FIG. 2 being administered to a patient to expose a phage of FIG. 1 associated with the microbe within the patient to the treatment electromagnetic field resonated by the solution.

FIG. 4 is a detailed view of the phage of FIG. 3 after being exposed to the treatment electromagnetic attacking the microbe.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

The invention disclosed herein is a method that provides a novel technology that enables the inducing of strategic and beneficial alterations of natural activities of native phages, or phages already present within the body, within vectors, or other organisms. These various benefits can exceed those of conventional antimicrobial medications and provide other physiologically advantageous treatments. The method is referred as the Induced Native Phage Therapy (INPT). The method is enabled by and incorporates a novel testing and treatment technology called Biospectroscopic Emission Sequencing (BES Technology). Developed by the Applicant, BES Technology enables the user to manually sequence biologically-generated, electromagnetic (EM) signatures into a form of EM sentences, which through the resonance and emission interactions between the one testing and that of the subjects, such as tested living organisms or tested humans, can assist in determining strategic bioinformation within the tested human or organism, as well as the determination of the specific EM-frequency signatures necessary to achieve the intended induction of the native phage to lytic activity.

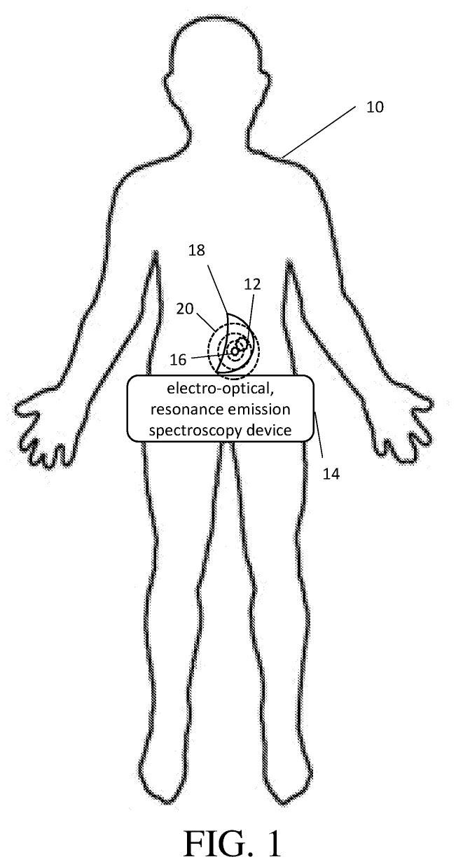

BES Technology is a complex biological electro-optical, resonance emission spectroscopy technique. FIG. 1 shows an enhanced form of testing to determine the presence of a target 12, such as a microbe, in a subject 10. The subject 10 in FIG. 1 is a human, but in other embodiments can be an animal, bug, food substance, water-treatment or other organism which may be exposed or carry infectious target cells. An electro-optical, resonance emission spectroscopy device 14 such as a zero-refractive index metamaterial block with a fitted polarizing filter can be placed or rested on or near the subject 10, and in a suspected area of infection. In FIG. 1, the microbe 12 is located in the subject's stomach 18, but the microbe 12 could be positioned in various locations within the body. The spectroscopy device 14 can be utilized to manipulate the biophotonic emissions in the subject 10 to access the emitted bioinformation produced as a result of the presence of the specific target 12 or microbe being identified in the subject 10. Certain DNA, including that of bacteria, can emit resonance EMF signatures in response to ambient EMF signatures in the environment. BES Technology can identify the microbe 12 by enabling frequency-matching of the detected primary microbial emission in the subject 10, determined by the microbe's emitted EM molecular signature 20 from the subject 10 to that of a known microbe specimen. Using BES Technology and/or conventional blood laboratory testing, the primary infection can be determined.

In one embodiment, EM signatures can be transmitted to the subject via a tester's fingers placed in various positions and orientations, hereinafter called hand modes, with the purpose of producing varying EM signatures. Each hand mode is associated with a particular EM signature. Frequency matching can occur when resonance is detected between the EM signature generated by the particular hand mode and the EM signature emitted from the target within the body. Such EM frequencies can also be generated via a microcurrent machine and electrodes placed in strategic positions on the subject, and a resonance can be detected as the EM signature provided by the microcurrent machine is varied to frequency match that of the target present in or on the subject. In other embodiments, target samples/specimen, such as samples of bacteria, can be placed on or in proximity to the subject being tested. If the target sample/specimen is present in the subject, resonance emissions can be detected between the target sample/specimen and the target in the subject to confirm the present of the target in the subject. This resonance information can be used to determine the primary target for treatment with INPT, and thereby also be used in the BES sequencing to the final treatment solution.

If the specimen is already known through conventional testing, BES can use a sample of that specimen to sequence the ultimate INPT treatment solution. The BES Technology results can also be confirmed by conventional testing, though BES Technology can offer the advantage over conventional testing that the primary type of microbial infection can be rapidly determined in real time, as opposed to lab testing where the tests have to be sent off for analysis, thus delaying the results and microbe 12 identification.

Further, BES technology format sequencing can then determine the complex and optimum energy of correction, or the specific triggering EMF signature that when exposed to natural or native phages 16 in the subject 10 that are associated with the identified microbe 12, induces the native phages 16 to target, via lytic swarming, the identified microbe 12, leading to its substantially complete and rapid elimination. “Substantial elimination” or “substantially complete elimination” means modifying the target cells or reducing a sufficient number of target cells within the subject to resist mutation of the target cells in response to the INPT treatment or prevent spreading or new growth of the target cells within the subject 10 after treatment. The optional use of a zero-refractive index, metamaterial in the BES technology permits more sensitive biophotonic light bioinformation from the test subject to be manipulated, to squeeze, bend, twist and/or reduce a diameter of a segment of biophoton information from the macroscale to the nanoscale in an effort to harness more bioinformation needed to specifically determine and target the triggering EMF signature required to activate a specific native phage 16 of the identified microbe 12 within the subject 10 to attack the microbe 12 via, but not limited to lytic activities. Major mechanisms of epigenetic gene regulation include environmental factors, such as the subtle EM signature of INPT, where gene expression is modified without involving changes in DNA sequence. In causing the lytic effect, the triggering EM signatures can cause redirection of the cellular expression of the phages by either indirect epigenetic regulation where cellular signaling or transcriptional dysregulation occurs or direct epigenetic regulation where epigenetic cofactors such as histone deacetylases are targeted. In viruses, transformation is a consequence of the expression of the viral latency proteins and RNAs which again can have either a direct or indirect effect on epigenetic regulation of cellular expression.

### The INPT Remedy

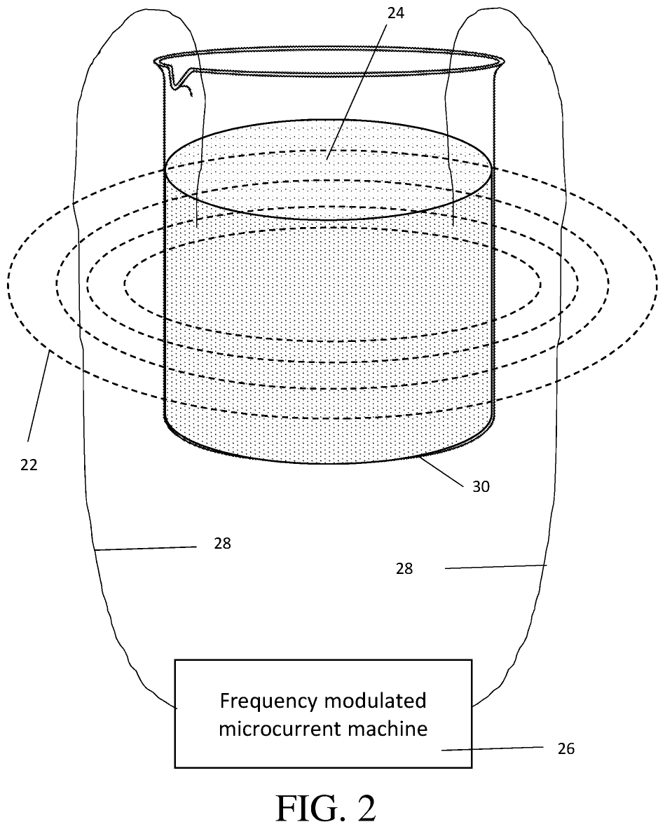

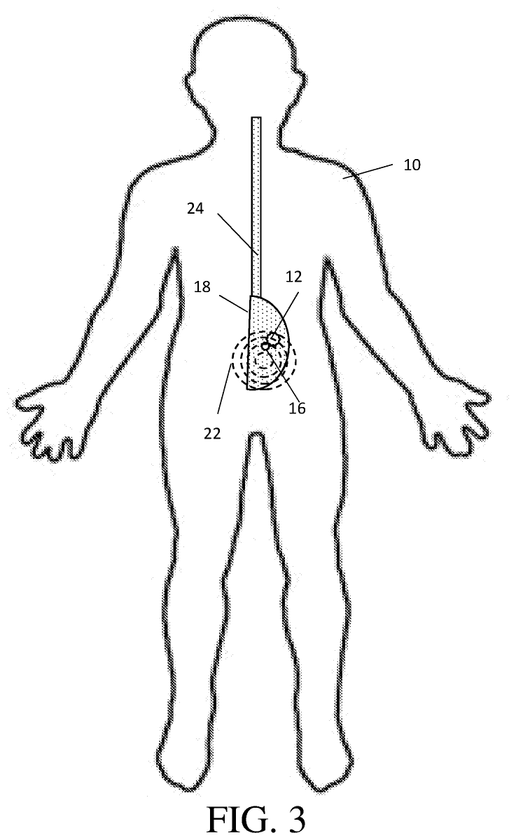

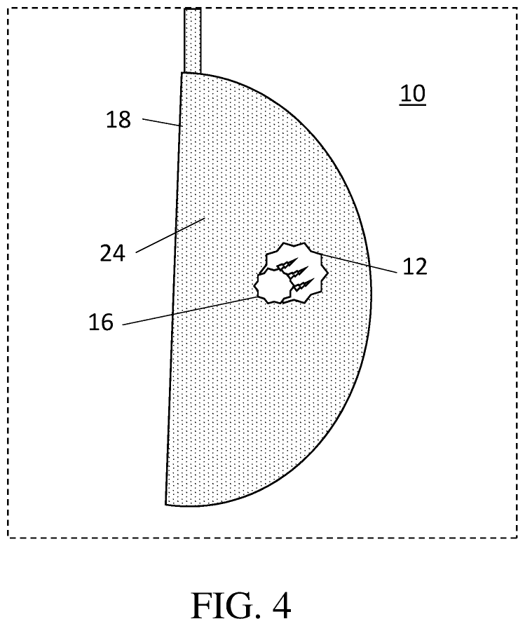

Once the triggering EMF signature is determined utilizing the BES Technology protocol, the method can further include, as shown in FIGS. 3-4, exposing the subject 10 to a treatment EMF 22 which has an EMF signature that is substantially the same as the triggering EMF signature. In some embodiments, as shown in FIGS. 2-3, the treatment EMF can be exposed to the subject 10 via a carrier medium 24 or aqueous solution in which the treatment EMF 22 can be generated or imprinted, such that the treatment EMF 22 can be propagated via the solution 24. The solution 24 in some embodiments can then be ingested by the subject 10 via pills containing the solution 24 or by drinking the solution 24, for instance, to expose the solution 24 to the subject and induce phages to eliminate the target or their host organism, possibly located in various locations within the body 18. The solution 24 in other embodiments can be administered orally, intravenously, through injection, by aerosol, or via a nebulizer to place the solution 24 within the subject 10 and near the identified microbe 12 and the host phage 16.

In some embodiments, the solution 24 propagating the treatment EMF 22 can be formed with the use of a frequency-modulated microcurrent machine 26. Leads 28 from the microcurrent machine 26 can be placed either directly in the solution to transmit an electrical current through the solution 24 from one lead to another, or by placing the solution in an electrically conductive container 30 such as borosilicate beaker and connecting the leads 30 via electrode pads on opposite sides of the conductive container 30, to induce electromagnetic energy to propagate from the solution 24. Desired settings to replicate the triggering EMF signature can be input into the microcurrent machine 26 depending upon the type of microbe 12 being targeted (e.g. current (311 μA), wave slope (gentle), polarity (bipolar), and frequency (11 Hz)). The microcurrent machine 26 can be turned on for a predetermined time period (e.g. 1, 2, 3, or 4 minutes, or any other suitable time period) to run electrical current through the solution or expose the solution 24 to an electromagnetic field from the current provided by the microcurrent machine 26, to imprint or generate in and propagate the desired treatment EMF 22 from the solution 24.

The practical application of BES Technology and INPT can be achieved as described above, using manual bioenergetic methods, but can also be achieved using various EM devices, such as, but not limited to a programmable, micro current (microampere current) devices (programmable for current (micro amps), wave slope, polarity, and frequency (Hz)).

Once the subject 10 is exposed to the solution 24 and the treatment EMF 22 is propagated from the solution 24, the treatment EMF 22 can cause a near instantaneous and systemic activation of the specific type of phage 16 that has been targeted to induce lytic killing of the microbe 12. Multiple treatment delivery options are possible for the solution 24 with the INPT technology, including, but not limited to, oral drops, injections, intravenous, sprays, nebulizer, or topical applications, etc. In other embodiments, an EMF generating device, such as an electromagnet or electromagnetic device, can be placed near the subject 10, set at desired settings to produce the treatment EMF 22, and run to generate and expose the subject 10 to the treatment EMF 22.

In one embodiment, the solution 24 or carrier medium can be a hydro-nanoparticle remedy, and can be formulated with a proprietary combination of calcinated Equisetum arvense and calcinated Avenae sativa, resulting in a dilute solution of plant-derived nanoparticle silica, gold, and other minerals, that are known to absorb and scatter light and therefore absorb and scatter the coherent, electro-photonic induction signature throughout the body, for the induction of native phages to alter their function towards the desired therapeutic goal. The energy of induction can be delivered via many types of carriers or modalities, as previously noted, with or without nanoparticles. The advantage of using nanoparticles is that they are very efficient at stabilizing the integrity or holding more strongly the imprinted EM signature and function to serve as efficient carriers and transmitters of the imprinted single or complex spectrum of microbe-specific, phage-inducing EM signatures, enabling an extended time of activity within the subject being treated. This can provide an added benefit over other treatment options, such as the use of electrical equipment, as the solution 24 can be administered to the subject 10 and be working over an extended period of time without the subject 10 having to continually be near or hooked up to electrical equipment.

Viruses (phages) can be activated from a dormant state by the effects of extremely low frequency electromagnetic fields (ELF-EMF). ELF-EMF fields are too low in strength to damage DNA or to promote genetic mutations in humans or animals. ELF-EMF, as applied in INPT is not designed to activate phages from a “dormant” state, since phages are actively engaged in lysogenic activities at all times, and INPT is specifically striving to precisely induce a change in that activity to lytic or other activities via EM-induced changes in the epigenetic switches influencing the genetic expression of the viral (phage) genomes, which is well-known to be sensitive to environmental conditions.

In some embodiments, the method can further include identifying one or more additional targets within the subject 10, and exposing the subject 10 to one or more additional corresponding treatment electromagnetic signatures, each of the one or more additional corresponding treatment electromagnetic signatures suitable to induce corresponding additional native phages in the subject 10 to attack the corresponding additional target, or advantageously modify the corresponding additional target pathology or substance. Similarly, in some embodiments, the solution 24 or other carrier medium can be imprinted with multiple treatment EMFs representing various triggering EMFs, the solution 24 or carrier medium propagating each target EMF when ingested by or exposed to the subject 10. Each target EMF can be imprinted in the solution 24 as described herein sequentially by running a first micro current through the solution 24 or carrier medium at a first set of desired settings for a first predetermined period of time, then running a second micro current through the solution 24 or carrier medium at a second set of desired settings for a second predetermined period of time, and so on. Imprinting the solution 24 or carrier medium with multiple treatment EMFs can allow a “broad spectrum” treatment protocol to be administered to the subject 10 that can effectively target and attack multiple target cells simultaneously. This can be beneficial for instance when a type of bacteria, virus, etc. is identified in the body, but it is difficult to determine which specific strain or species of that target is present in the body, or when the type of bacteria is not readily identifiable, but the clinician can narrow the potential culprit of the patient's ailments to a several different types of target cells.

### Documentation of the Effectiveness of INPT

Induced Native Phage Therapy (INPT) has been used to experimentally target dozens of types of microbes in an estimated 280 chronically ill people, with never before seen results.

### Borrelia sp.

Of these 280 people, in an IRB supported retrospective registry study, 60 qPCR Borrelia-phage blood tests were performed on almost 30 people with previously diagnosed, non-responsive Borrelia sp. spirochete infections. A before and after Borrelia-Phage test was used to first document that they did indeed have the Borrelia infection. A Borrelia-targeted INPT treatment was administered orally two-three times a day for the first five days. Another Borrelia-phage test was performed two weeks after the initial test, allowing 7 days for the Borrelia-phages to die, due to having killed all of their hosts, which were needed to replicate more of the Borrelia phages. The results of the second test showed that in the people who were positive just two weeks prior, that greater than 95% of the people were now negative for any residual Borrelia. A third Borrelia-phage test was performed between 45-60 days after the second test to confirm long term elimination of the Borrelia. Of the people who were negative on their second test, 100% of them were still negative. If there had been any persistent Borrelia anywhere in the body, the 45-60 day window of time would have allowed the bacteria time for several replication cycles and the test would once again be positive. Thankfully this was not the case. Clinical-symptomatic improvements were often above expectations, while others who had suffered from years of damage to their tissues as a result of the many failed treatments and from the illness itself, experienced slight to significant improvements in clinical symptoms, though the Borrelia infection was successfully treated using the INPT treatment method.

Of the people with Borrelia infections treated with INPT, the group reported that they experienced none or mild worsening of symptoms, in spite of the speed of Borrelia elimination. The reason for the rapid elimination and minimal “herx” reactions is due to the speed and manner in which the phage kill the bacteria, resulting in there being no cytokines released from the body's immune cells. INPT appears to be able to target Borrelia and substantially eliminate it within two weeks, with little to no toxic and potentially harmful effects, which are common with all antibiotics, as documented by the newest and most sensitive Borrelia testing, the Phelix Borrelia-Phage Test.

### Taenia Solium (Pork Tapeworm) Versus Paraphage

In a subject with detected Taenia solium tapeworms, INPT was implemented on a trial basis to determine whether native phages could be caused to target and kill the tapeworms, via lytic activity or other undetermined activities of the phages. Within two hours after the initiation of a Taenia-specific INPT remedy the subject reported that her abdomen bloated slightly with feeling of tapping against the abdominal wall, which was thought by the subject to be the worms flipping about. Three hours later the subject self-administered an enema, with which the subject eliminated two dead, 1-1.5-foot tapeworms. The subject took a picture of the worms in the toilet. From the picture it appeared that the Taenia-specific phages had attacked the large worms from all sides in a lytic swarm, and killed them within five hours of the one dose of the INPT remedy.

### MRSA versus INPT Bacteriophage

Methicillin-resistant Staphylococcus aureus (MRSA) is well-known to be treatment resistant to all antibiotics. A subject with longstanding, recurrent MRSA infection on his scalp, which when flared would normally require several types of treatments and weeks to heal, was treated with INPT targeting a variety of strains of Staphylococcal bacteria. Within 24 hours the skin eruption healed and in a few days the event was unnoticeable.

### Bartonella-Infected Tick versus INPT

Ticks which were infected with Bartonella bacteria were topically treated with one drop of an INPT Bartonella-phage inducing remedy, with the idea that due to the fact that the INPT effect is its unique electromagnetic signature, its frequencies would easily penetrate the tick, inducing the Bartonella-phages already using the Bartonella bacteria as a lysogenic host, to switch to lytic actions, killing all of the Bartonella bacteria inside the tick. Within one hour none of the Bartonella bacteria were detectable when retested. A controlled laboratory study is in process to confirm the therapeutic effects of the INPT remedy as applied topically to ticks and other infections carrying insects.

It is expected, based upon the tick study, that aerial spraying of desired areas, such as outdoor areas, yards, parks, etc. where certain disease carrying bugs are prevalent, including but limited to ticks and mosquitos, etc., with the appropriate INPT solution may help fight the spread and infection of vector-borne diseases, such as bartonella, Lyme disease, tularemia, babesiosis, malaria (Plasmodium vivax, malariae, and falciparum), and other insect-borne illnesses. INPT used in this manner can help prevent the spread of such diseases by killing the disease at the source, the biting insects. This study on ticks demonstrates that the use of INPT in this manner can be a novel and ecologically friendly approach, in that vector-borne microbes can be targeted and killed without killing the host insect, and without the use of broad-spectrum pesticides.

### Aspergillus and Penicillium Mold Versus INPT

An article of clothing, smelling strongly of mold was sprayed with a mixed-strain Aspergillus and Penicillium INPT remedy. When the shirt dried from the spray, it no longer smelled musty. In another test, an INPT cocktail of phage-inducers targeting the most common household mold, penicillium spp., Aspergillus, and others, were fogged inside one level of a mold contaminated home, leaving the other level unfogged. Subsequent mold testing by independent mold inspection, determined that the spore counts for the fogged room were several magnitudes less than that of the non-fogged level of the house. Although, this test is somewhat anecdotal, it provides strong proof of concept that the INPT can be used to treat and kill mold.

### Improvements over Conventional Antimicrobial Medications

Testing utilizing the INPT treatment show that the treatment has been well-tolerated, even by people who are sensitive to almost all medicines, making it ideal for those with Multiple Chemical Sensitivity (MCS), Mast Cell Activation Syndrome (MCAS), and other hypersensitivity conditions.

The manner in which phages kill a microbe causes much less “die-off” worsening of a person's symptoms than antibiotics and other antimicrobial products. We now know from research that a Jarisch-Herxheimer “Herx” reaction is not caused by the bacterial debris (endotoxins) spilling into the body as the bacteria are killed as with conventional antibiotics. A Herx reaction is now understood to be a reaction to proinflammatory cytokines released by the body's immune cells that phagocytize dying bacteria, and is common due to the unique manner in which conventional pharmaceutical antibiotics kill bacteria. With phage lysing of the bacteria, from INPT stimulation, the bacteria (microbe) explodes where it sits and only the debris is cleaned up by the immune cells, with no release of cytokines by the immune cells. The debris does not cause much of a proinflammatory cytokine release and therefore any “Herx” reaction associate with the INPT treatment is minimal. Thus, the INPT treatment can help provide rapid and complete annihilation of the target microbe, while reducing any negative “Herx” reaction symptoms in the patient.

Conventional antibiotics are extensively documented as causing severe Jarisch-Herxheimer (JH) reactions, leading many people to experience new and often extreme worsening of their symptoms, on top of their original symptoms, with some experiencing such worsening of their symptoms that they are even driven to suicide. Due to the speed and mechanism of killing of the target host, utilized by native phages in lytic activities, there are minimal proinflammatory cytokines released and therefore very minimal JH reaction.

Additionally, unlike once thought, antibiotics are not a viable solution for those with recurrent, longstanding or chronic infections, such as Methicillin-Resistant Staphylococcus Aureus (MRSA) and Lyme disease. The fact that INPT can, in some instances, according to sensitive qPCR testing, completely eliminate all of the targeted bacterial population, within two weeks of treatment, and do it without causing severe worsening of the patient's symptoms is a significant improvement over antibiotics.

Induced Native Phage Therapy (INPT) provides various benefits over conventional antimicrobial medications and other physiologically advantageous treatments. Being a complex frequency-based remedy, with no chemical constituents, it is considered in therapeutic dosing to be non-toxic and non-allergenic, and generally well-tolerated. With no chemical constituents, the sterile nature of the INPT delivery method, can help prevent compound degradation, that would otherwise shorten the product's shelf-life. The INPT treatment is stable in normal ambient light and temperatures from −29° C. to 55° C. INPT is sensitive to strong extraneous sources of electromagnetic radiation, such as close proximity to microwaves, cell phones, and computers, in addition to other strong EMF contaminating sources. When using INPT to induce phages to target and attack an infection, the induced phages are stimulated to switch from lysogenic activities to lytic activities in response to subtle changes in their environment. INPT is a new invention that uses complex, subtle electromagnetic frequencies to induce this change in phages that entered the body with the infection and are specific to their host microbe/organism, thus eliminating the need to match the subject's infection to an externally-sourced or genetically-engineered cocktail of phages and then introducing these matched phages into the body, which is not currently an approved form of phage treatment in many countries. As with all viruses, phages need a host in order to propagate more phages. Without a host, INPT induced-phages will themselves die within a week of the death of the last host microbe/organism, as there will be no more of the hosts, which are necessary to replicate more of that type of phage. Therefore, the INPT induced phages exert minimal residual effects on the body. Both humoral and innate immune components help clear residual debris of the dead hosts from the body.

If a person is re-infected through vector-borne transmission, INPT induced-phages will work as effectively as during the prior infection, as the microbe involved with the infection, will enter the body already parasitized by its phage-type from nature. These native phages will have never experienced INPT induction, and therefore the treatment will work as anticipated. The complete annihilation of the targeted microbe, due to its inability to resist or adapt to the phage attacks, insures against the host from mutating and becoming phage-resistent, as is commonly seen in microbes building resistance or adapting to an antibiotic or vaccine treatment. The INPT treatment has a relative low cost of treatment and short duration of treatment of five days of treatment, with the possible need for a booster round of another five days. An unlimited variety of targets such as microbes/organisms can be quickly and conveniently targeted using the INPT method. The INPT treatment can only affect the targeted type or types of bacteria, leaving friendly flora unaffected. Phages are known to cross the blood-brain-barrier to reduce or substantially eliminate brain infections. The INPT treatment provides little to no ability of the targeted microbe/organism to defend against the induced-phage attacks. Multiple strains and species of microbe can be targeted at the same time using a suitable treatment EMF selection. The INPT remedy utilizes a parallel but somewhat cooperative regulation of microbes with the humeral immune system. Rapid elimination of targeted microbe, with some targeted organisms being apparently fully eliminated within 48 hours. INPT-induced phages auto-dose as their host microbes are killed, releasing prophages from within the host organism. Organisms which are swarmed by lytic phages are also unable to regain their viability. The INPT treatment has been shown, by laboratory testing to apparently completely eradicate bacteria, in a retrospective study of INPT treatment of people with qPCR Borrelia-Phage testing confirmed Borrelia, Borrelia-persistor cells, and different strains of Borrelia sp., typically in less than seven days. INPT induced phages can also penetrate bacterial biofilms to reach hidden target bacteria within the subject.

The INPT treatment does not require a healthy immune system, although phage-immune interactions can occur. The INPT treatment does not have any known significant environmental impact, as opposed to the negative effects of pharmaceutical antimicrobial medications which can remain biologically active in water treatment facilities, and tapwater, ground water, and in the food supply. The INPT treatment does not alter phages in the environment, due to the fact that the INPT effects are in situ.

INPT offers a range of uses including, but not limited to: antibacterial treatment (Bacteriophage-Inducer), anti-fungal treatment (Mycophage-Inducer), Anti-parasitic treatment (Paraphage-Inducer), as wells as therapeutically INPT can also be used to target and attack or modify pathological targets, or eliminate or modify biologically/physiologically advantageous targets.

The external application (Lawn, parks, acreage) of INPT solution to kill the infectious microbes common to ticks, mosquitos, and other vectors of infections. This will enable an environmentally friendly way to kill the pathogenic microbes living within a host carrier, as the host is not killed, and there is no toxic poison. The external application of an INPT solution can help prevent birds, animals, and humans which eat the host or who may be bitten by the hosts or insects from becoming infected with the undesirable microbe. The INPT phage-inducer will, when sprayed on the insect cause an immediate activation of the phages already active within the insect to switch from lysogenic activities to lytic swarming, killing all of the targeted pathogenic bacteria living within the insect. When a human or animal is bitten by the insect, no infectious bacteria will be injected with the bite.

Mold in buildings, in laundry machines, and on garments can be killed by applying an INPT solution designed to address the common types of fungi found in such locations.

Phage-inducers can be utilized for inducing phages for the removal of harmful accumulations of biological substances, such as Beta-Amyloid plaques in the brain, in such conditions as Alzheimer's disease, Parkinson's, and Multiple Sclerosis. Phage-inducers can also be utilized for the targeting of abnormal cell replication,

INPT can be utilized for water sterilization or treatment for common microbes found in water sources, and for sanitizing surfaces.

Thus, although there have been described particular embodiments of the present invention of a new and useful Method Of Manipulating Phages Within The Body To Attack Viral And Bacterial Cells, it is not intended that such references be construed as limitations upon the scope of this invention.

What is claimed is:

1. A method of regulating, manipulating or inducing a change of activity, from lysogenic to lytic activities in native or naturally occurring phages within a subject to treat a bacterial, viral, fungal, or parasitic infection, or other pathological or biologically-physiologically advantageous target in the subject, the method comprising the steps of:
identifying the target within the subject;
and
exposing the subject to a treatment electromagnetic signature that is suitable to induce the native phages in the subject to attack the target, or advantageously modify the target pathology or substance.

2. The method of claim 1, wherein exposing the subject to the treatment electromagnetic field further comprises:
providing an aqueous solution;
passing either a specific selection of biologically or mechanically-generated electromagnetic currents through the solution for a predetermined amount of time, to imprint the treatment electromagnetic signature onto and into the solution as a carrier, to cause the solution to carry and propagate the treatment electromagnetic signature from the solution to the subject, and
administering the solution to the subject.

3. The method of claim 2, wherein the aqueous solution contains silica, gold and/or other metal nanoparticles.

4. The method of claim 2, wherein the aqueous solution comprises a saline solution.

5. The method of claim 2, wherein the aqueous solution is delivered either orally, intravenously, through injection, by aerosol, or via a nebulizer directly onto or into the subject.

6. The method of claim 1, wherein exposing the subject to a treatment electromagnetic signature comprises:
generating the treatment electromagnetic signature with an electromagnetic signature generation device; and
positioning the electromagnetic signature generation device adjacent the subject.

7. The method of claim 1, wherein the target is chosen from a Borrelia infection, a Staphylococcus infection, a Bartonella infection, an Asperigillus infection, and tape worm.

8. The method of claim 1, wherein the bacterial infection is a chronic Borrelia burgdorferi infection.

9. The method of claim 1, wherein exposing the subject to a treatment electromagnetic signature cause the native phages to substantially eliminate the target.

10. The method of claim 1, wherein the subject is one of ticks, mosquitos, or rodents.

11. The method of claim 1, further comprising:
identifying the target within the subject and determining a triggering electromagnetic signature suitable to induce the native phages in the subject to attack the target, or advantageously modify the target pathology or substance utilizing biological electro-optical, resonance emission spectroscopy; and
matching the treatment electromagnetic signature to the triggering electromagnetic signature.

12. The method of claim 1, further comprising:
identifying one or more additional targets within the subject; exposing the subject to one or more additional corresponding treatment electromagnetic signatures, each of the one or more additional corresponding treatment electromagnetic signatures suitable to induce corresponding additional native phages in the subject to attack the corresponding additional target, or advantageously modify the corresponding additional target pathology or substance.

13. A method of regulating, manipulating or inducing a change of activity, from lysogenic to lytic activities in native or naturally occurring phages within a subject to treat a bacterial, viral, fungal, or parasitic infection, or other pathological or biologically-physiologically advantageous potential target in the subject, the method comprising the steps of:
identifying the potential target within the subject;
implanting a treatment electromagnetic signature into an ingestible carrier medium, the treatment electromagnetic signature suitable to induce the native phages in the subject to attack the potential target, or advantageously modify the potential target pathology or substance; and
providing the ingestible carrier medium to the subject to ingest to induce the native phages in the subject to attack the target or modify the target pathology or substance.

14. The method of claim 13, further comprising:
identifying one or more additional potential targets within the subject;
implanting one or more corresponding plurality of treatment electromagnetic signatures into the same ingestible carrier medium, each of the one or more corresponding treatment electromagnetic signatures suitable to induce corresponding native phages in the subject to attack the corresponding potential target, or advantageously modify the corresponding potential target pathology or substance.

15. The method of claim 13, wherein the ingestible carrier medium is a saline solution containing one or more types of metal nanoparticles.

16. The method of claim 15, wherein the one or more types of metal nanoparticles includes silica and/or gold.

17. A method of regulating, manipulating or inducing a change of activity, from lysogenic to lytic activities in native or naturally occurring phages within a subject to treat a bacterial, viral, fungal, or parasitic infection, or other pathological or biologically-physiologically advantageous target in the subject, the method comprising the steps of:
identifying the target within the subject;
determining a triggering electromagnetic signature that, when exposed to native phages within the subject associated with the target, induces the native phages, which are already within the subject, to seek out and attack or modify the target via lytic swarming; and
exposing the subject to a treatment electromagnetic signature having that is substantially the same as the triggering electromagnetic signature that is suitable to induce the native phages in the subject to eliminate the target, or advantageously modify the target pathology or substance.
